(19) United States
(12) Patent Application Publication
(10) Pub. No.: US 2023/0000408 A1
(43) Pub. Date: Jan. 5, 2023

(54) Blood Collection Device and Related Systems and Methods

(71) Applicant: Becton, Dickinson and Company, Franklin Lakes, NJ (US)

(72) Inventors: Yiping Ma, Layton, UT (US); Jonathan Karl Burkholz, Salt Lake City, UT (US)

(21) Appl. No.: 17/852,553

(22) Filed: Jun. 29, 2022

Related U.S. Application Data
(60) Provisional application No. 63/218,101, filed on Jul. 2, 2021.

(51) Int. Cl. A61B 5/15

(52) U.S. Cl. CPC A61B 5/150992; A61M 2025/09083

(57) ABSTRACT

A blood collection device may be configured to couple to a catheter assembly. The blood collection device may include a housing, which may include a distal end, a proximal end, and a slot. The blood collection device may include a tube disposed within the housing. The blood collection device may include an advancement element configured to move along the slot. In response to distal movement of the advancement element along the slot, the tube may be configured to advance distally beyond the distal end of the housing. The instrument advancement device may include a coil extending distal to the tube.

### CROSS-REFERENCE TO RELATED APPLICATION

The present application claims priority to U.S. Provisional Application Ser. No. 63/218,101, entitled “Blood Collection Device and Related System and Methods”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond a distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increase. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to blood collection devices and related systems and methods. In some embodiments, a blood collection device may be configured to couple to a catheter assembly. In some embodiments, the blood collection device may include a housing, which may include a distal end, a proximal end, and a slot. In some embodiments, the blood collection device may include a tube disposed within the housing. In some embodiments, the blood collection device may include an advancement element configured to move along the slot. In some embodiments, in response to distal movement of the advancement element along the slot, the tube may be configured to advance distally beyond the distal end of the housing.

In some embodiments, the blood collection device may include a coil extending distal to the tube. In some embodiments, the coil may be coupled to a distal end of the tube. In some embodiments, the blood collection device may include a core wire disposed within the tube. In some embodiments, the core wire may be coupled to the coil and may extend proximally from the coil to the advancement element. In some embodiments, the coil may extend into the tube. In some embodiments, a proximal end of the coil may be distal to a distal end of the tube, and a distal end of the core wire may be distal to the distal end of the tube.

In some embodiments, the tube may include multiple sections. In some embodiments, the sections may include a first section and a second section proximal to the first section. In some embodiments, the first section may be a distalmost section of the sections. In some embodiments, the first section may include a first inner diameter and a first outer diameter that may be constant along the first section. In some embodiments, the second section may include a second inner diameter and a second outer diameter that may be constant along the second section. In some embodiments, the coil may extend distal to the first section. In some embodiments, a proximal end of the coil may be coupled to the first section.

In some embodiments, a catheter system may include the blood collection device. In some embodiments, the catheter system may include a catheter assembly, which may include a catheter. In some embodiments, the catheter may include a distal tip and a proximal end. In some embodiments, in response to movement of the advancement element along the slot from the retracted position to the advanced position, the first section may extend through the distal tip of the catheter and the second section may be at least partially disposed within the catheter.

In some embodiments, the blood collection device may include a septum. In some embodiments, in response to movement of the advancement element along the slot from the retracted position to the advanced position, the first section and the second section may move through the septum. In some embodiments, the catheter assembly may include a catheter adapter, which may include a distal end, a proximal end, a lumen extending through the distal end of the catheter adapter and the proximal end of the catheter adapter, and a side port between the distal end of the catheter adapter and the proximal end of the catheter adapter and in fluid communication with the lumen. In some embodiments, the catheter assembly may include an extension tube integrated within the side port.

In some embodiments, in response to movement of the advancement element along the slot from the retracted position to the advanced position, a distal end of the first section may be disposed within the extension tube or within the side port distal to the extension tube. In some embodiments, in response to movement of the advancement element along the slot from the retracted position to the advanced position, a distal end of the first section may be disposed within the catheter. In some embodiments, in response to movement of the advancement element along the slot from the retracted position to the advanced position, a distal end of the first section may be disposed within the catheter adapter proximal to the catheter.

In some embodiments, the catheter assembly may include a wedge securing the catheter within the catheter adapter. In some embodiments, in response to movement of the advancement element along the slot from the retracted position to the advanced position, the distal end of the first section may be disposed within the wedge proximal to the catheter.

In some embodiments, another blood collection device may be configured to couple to the catheter assembly or another suitable catheter assembly. In some embodiments, the other blood collection device may include a housing, which may include a distal end, a proximal end, and a slot. In some embodiments, the other blood collection device may include a tube disposed within the housing and a guidewire comprising a coil. In some embodiments, a distal advancement element may be configured to move along the slot and coupled to the tube. In some embodiments, in response to distal movement of the distal advancement element along the slot, the tube may be configured to advance distally beyond the distal end of the housing.

In some embodiments, a proximal advancement element may be configured to move along the slot and coupled to the guidewire. In some embodiments, in response to distal movement of the proximal advancement element along the slot, the coil may be configured to advance distally beyond the distal end of the housing. In some embodiments, when the distal advancement element and the proximal advancement element are each in an initial position, the distal advancement element may be distal and spaced apart from the proximal advancement element.

In some embodiments, the proximal advancement element may be configured to move distally along the slot a first distance from the initial position to contact the distal advancement element. In some embodiments, in response to movement of the proximal advancement element distally along the slot a second distance greater than the first distance from the initial position, the distal advancement element and the proximal advancement element move distally along the slot together.

In some embodiments, the tube may include the sections, which may include the first section and the second section proximal to the first section. In some embodiments, the first section may be a distalmost section of the sections. In some embodiments, the first section may include the first inner diameter and the first outer diameter that may be constant along the first section. In some embodiments, the second section may include the second inner diameter and the second outer diameter that may be constant along the second section. In some embodiments, in response to distal movement of the distal advancement element along the slot, the first section and the second section may be configured to advance distally beyond the distal end of the housing.

It is to be understood that both the foregoing general description and the following detailed description are examples and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of a blood collection device, illustrating an example advancement element in an example retracted position, according to some embodiments;

FIG. 1B is a cross-sectional view of the blood collection device, illustrating the advancement element in the retracted position, according to some embodiments;

FIG. 1C is an upper perspective view of an example catheter system that includes the blood collection device, illustrating the advancement element in the retracted position, according to some embodiments;

FIG. 1D is a cross-sectional view of a distal portion of the catheter system when the advancement element is in the advanced position, according to some embodiments;

FIG. 1E is an enlarged cross-sectional view of the distal portion of the catheter system when the advancement element is in the advanced position, illustrating an example coil with an example blunt end, according to some embodiments;

FIG. 1F is an enlarged cross-sectional view of the distal portion of the catheter system when the advancement element is in the advanced position, illustrating an example coil with another example blunt end, according to some embodiments;

FIG. 2 is a schematic diagram of the blood collection device, according to some embodiments;

FIG. 3A is a cross-sectional view of another catheter assembly, illustrating an example tube of the blood collection device when the advancement element is in the advanced position, according to some embodiments;

FIG. 3B is a cross-sectional view of the other catheter assembly, illustrating another example tube of the blood collection device when the advancement element is in the advanced position, according to some embodiments;

FIG. 3C is a cross-sectional view of the other catheter assembly, illustrating another example tube of the blood collection device when the advancement element is in the advanced position, according to some embodiments;

FIG. 3D is a cross-sectional view of the other catheter assembly, illustrating another example tube of the blood collection device when the advancement element is in the advanced position, according to some embodiments;

FIG. 4A is a schematic diagram of the blood collection device, illustrating an example core wire, according to some embodiments;

FIG. 4B is a cross-sectional view of the blood collection device coupled to the other catheter assembly, illustrating the example core wire when the advancement element is the advanced position, according to some embodiments;

FIG. 5A is an upper perspective view of another blood collection device, illustrating an example distal advancement element and an example proximal advancement element in an advanced position, according to some embodiments;

FIG. 5B is a schematic diagram of the other blood collection device, according to some embodiments;

FIG. 6A is a schematic diagram of the other blood collection device, illustrating the distal advancement element and the proximal advancement element in an example first configuration, according to some embodiments;

FIG. 6B is a schematic diagram of the other blood collection device, illustrating the distal advancement element in an example second configuration, according to some embodiments;

FIG. 6C is a schematic diagram of the other blood collection device, illustrating the distal advancement element in the advanced position and the second advancement element in an example third configuration, according to some embodiments;

FIG. 7A is a schematic diagram of the other blood collection device, illustrating the distal advancement element and the proximal advancement element in the first configuration, according to some embodiments;

FIG. 7B is a schematic diagram of the other blood collection device, illustrating the distal advancement element in another example second configuration, according to some embodiments; and

FIG. 7C is a schematic diagram of the other blood collection device, illustrating the distal advancement element in the advanced position and the second advancement element in another example third configuration, according to some embodiments.

### DETAILED DESCRIPTION

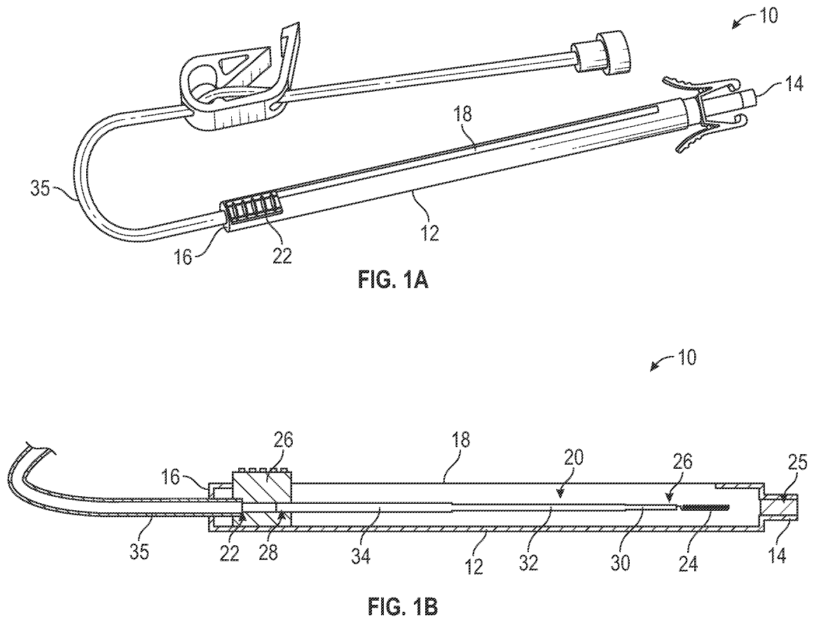

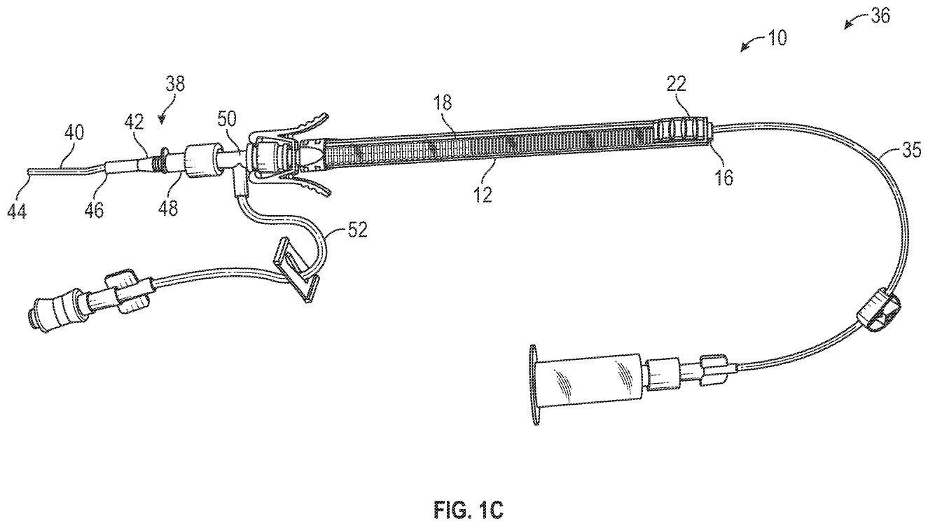

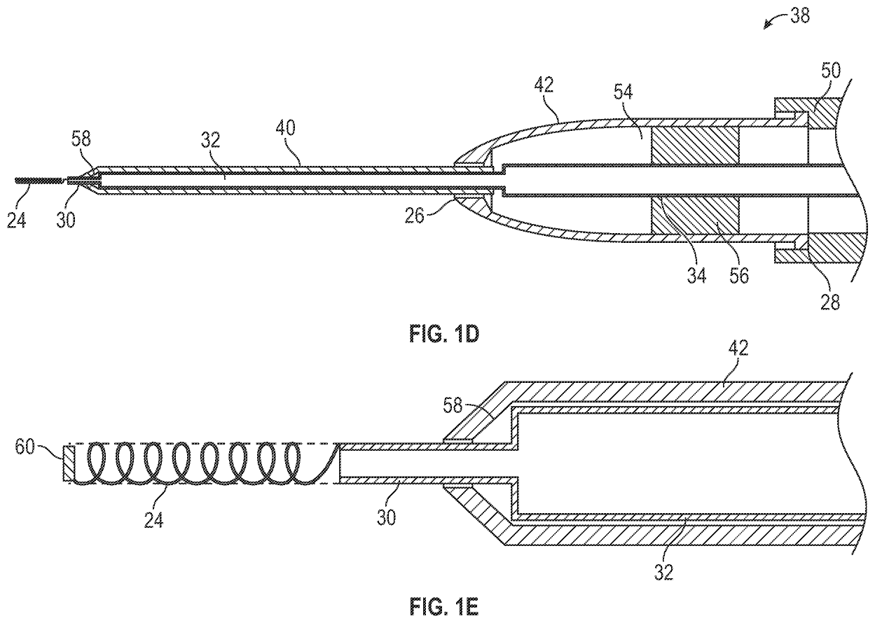

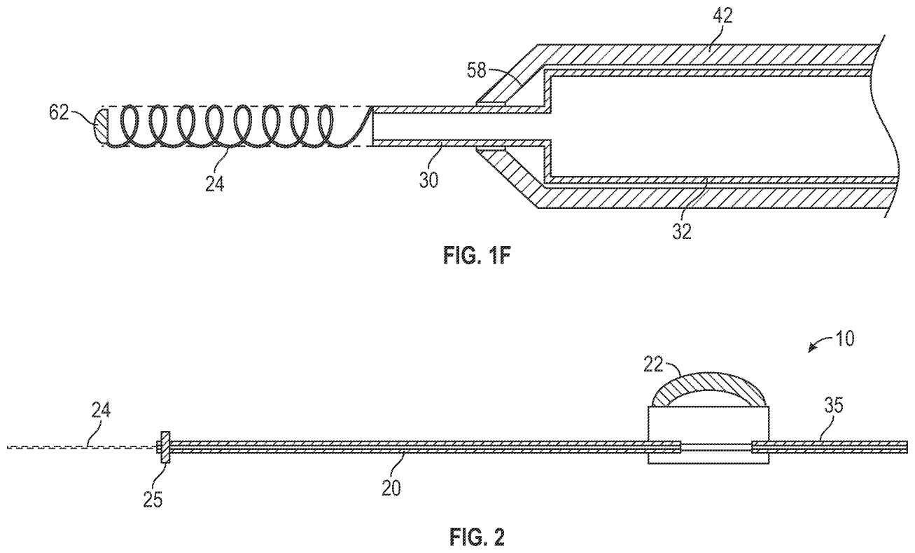

The present disclosure relates generally to blood collection devices and related systems and methods. Referring now to FIGS. 1A-1B, in some embodiments, a blood collection device 10 may be configured to couple to a catheter assembly that includes a catheter. In some embodiments, the blood collection device 10 may include a housing 12, which may include a distal end 14, a proximal end 16, and a slot 18. In some embodiments, the blood collection device 10 may include a tube 20 disposed within the housing 12. In some embodiments, the blood collection device 10 may include an advancement element 22 configured to move along the slot 18. In some embodiments, in response to distal movement of the advancement element 22 along the slot 18, the tube 20 may be configured to advance distally beyond the distal end 14 of the housing 12. In some embodiments, the tube 20 may be rigid, semi-rigid, or flexible.

In some embodiments, the blood collection device 10 may include a coil 24 extending distal to the tube 20. In some embodiments, the tube 20 may include a distal end 26 and a proximal end 28. In some embodiments, the coil 24 may be coupled to the distal end 26 of the tube 20, as illustrated, for example, in FIG. 1B. In some embodiments, the coil 24 may be constructed of metal, nitinol, or another suitable material. In some embodiments, the blood collection device 10 may include a septum 25, which may seal the distal end 14 of the housing 12.

In some embodiments, the tube 20 may include multiple sections. In some embodiments, the sections may include a first section 30 and a second section 32 proximal to the first section 30. In some embodiments, the first section 30 may be a distalmost section of the sections. In some embodiments, the first section 30 may include a first inner diameter and a first outer diameter. In some embodiments, the first inner diameter and the first outer diameter may be constant along the first section 30, such as along an entirety of the first section 30. In some embodiments, the second section 32 may include a second inner diameter and a second outer diameter. In some embodiments, the second inner diameter and the second outer diameter may be constant along the second section 32, such as along an entirety of the second section 32. In some embodiments, in response to movement of the advancement element 22 along the slot 18 from the retracted position to the advanced position, one or more of the first section 30, the second section 32, and the coil 24 may move through the septum 25.

In some embodiments, the sections may include one or more additional sections to the first section 30 and the second section 32. For example, the sections may include a third section 34 proximal to the second section 32. In some embodiments, the third section 34 may include a third inner diameter and a third outer diameter that may be constant along the third section 34, such as along an entirety of the third section 34.

In some embodiments, the sections may be immediately separated by a transition portion, such as a stepped surface or a tapered portion, which may transition from one inner and outer diameter of a particular section to another inner and outer diameter of another particular section. In further detail, in some embodiments, a particular transition portion may extend from the first section 30 to the second section 32, which may be adjacent to each other. In some embodiments, the second section 32 may be adjacent to the first section 30 and also to the third section 34. In some embodiments, another of the transition portions may extend from the second section 32 to the third section 36.

In some embodiments, the tube 20 may have a uniform inner diameter and/or a uniform outer diameter along an entirety of its length. However, in other embodiments, having multiple of the sections may facilitate a faster blood collection time through the tube 20 by providing an increased inner diameter and blood flow where an inner diameter of the catheter assembly allows. In some embodiments, the tube 20 may allow the user to draw a blood sample or infuse fluid through the catheter when the catheter is no longer functional or safe due to, for example, debris or thrombus build up on the distal tip of the catheter and/or collapse of the catheter. Thus, in some embodiments, the tube 20 may reduce a number of needle sticks that the patient experiences because the catheter may be replaced less frequently. In some embodiments, delivery of the tube 20 into the catheter assembly and/or the vasculature of the patient may reduce blood hemolysis and a risk of kinking of the catheter.

In some embodiments, the coil 24 may extend distal to the first section 30. In some embodiments, a proximal end of the coil 24 may be coupled or attached to the first section 30. In some embodiments, the coil 24 may prevent the debris or thrombus from forming on the distal tip of the catheter and blocking a fluid pathway during blood collection, thus improving an ability to draw blood.

In some embodiments, an extension tube 35 may be coupled to the advancement element 22 and in fluid communication with the tube 20. In some embodiments, a blood collection pathway may extend through the tube 20, the advancement element 22, and the extension tube 35. In some embodiments, the extension tube 35 and the proximal end 28 of the tube 20 may be sealed within the advancement element 22 to prevent blood from flowing into the housing 12. In some embodiments, the extension tube 35 may extend through the proximal end 16 of the housing 12 and may move through the proximal end 16 of the housing 12 in response to the advancement element 22 being moved along the slot 18.

Referring now to FIG. 1C, in some embodiments, a catheter system 36 may include the blood collection device 10. In some embodiments, the catheter system 36 may also include a catheter assembly 38, which may include a catheter 40 extending distally from a catheter adapter 42. In some embodiments, the catheter 40 may include a peripheral intravenous catheter, a midline catheter, or a peripherally-inserted central catheter. In some embodiments, the blood collection device 10 may be coupled to the catheter assembly 38 and the advancement element 22 may be moved from a retracted position distally to an advanced position, which may insert the tube 20 into the catheter assembly 38 and/or through the catheter 40 into the vasculature of the patient.

In some embodiments, the retracted position of the advancement element 22 may correspond to an initial position or the advancement element 22 may be returned to the retracted position after being moved to the advanced position. In some embodiments, the retracted position of the advancement element 22 may correspond to a fully retracted position in which the advancement element 22 is at or contacts a proximal end of the slot 18. In some embodiments, the advanced position of the advancement element 22 may correspond to a fully advanced position in which the advancement element 22 is at or contacts a distal end of the slot 18.

In some embodiments, the catheter 40 may include a distal tip 44 and a proximal end secured within the catheter adapter 42. In some embodiments, the catheter adapter 42 may include a distal end 46, a proximal end 48, and a lumen extending through the distal end 46 and the proximal end 48. In some embodiments, the blood collection device 10 may be directly coupled to the catheter adapter 42. In other embodiments, the blood collection device 10 may be coupled to the catheter adapter 42 via one or more connectors. For example, the catheter assembly 38 may include a T-connector 50 and/or a needleless connector, which may be disposed between the blood collection device 10 and the catheter adapter 42. In some embodiments, an extension tube 52 may be integrated with a port of the T-connector 50 and may be used for infusion of fluid into a patient.

In some embodiments, the catheter assembly 38 may include or correspond to any suitable catheter assembly, such as, for example, the BD NEXIVA™ Closed IV Catheter system, the BD CATHENA™ Catheter system, the BD VENFLON™ Pro Safely Shielded IV Catheter system, the BD NEOFLON™ IV Cannula system, the BD INSYTE™ AUTOGUARD™ BC Shielded IV Catheter system, or another suitable catheter assembly. In some embodiments, a proximal end of the extension tube 35 may be coupled to a BD VACUTAINER® one-use holder (available from Becton Dickinson & Company of Franklin Lakes, N.J.), a syringe, or any suitable adapter configured to receive a blood collection container such as a test tube or other suitable container.

Referring now to FIGS. 1D-1F, in some embodiments, in response to movement of the advancement element 22 along the slot 18 from the retracted position to the advanced position, the first section 30 may extend through the distal tip 44, and the second section 32 may be at least partially disposed within the catheter 40.

In some embodiments, the lumen 54 of the catheter adapter 42 may include a septum 56, through which the coil 24 and the tube 20 may extend in response to coupling the blood collection device 10 to the catheter assembly 38 and moving the advancement element 22 along the slot from the retracted position to the advanced position.

In some embodiments, the distal tip 44 of the catheter 40 may include a distal taper 58, which may contact the second section 32 in response to coupling the blood collection device 10 to the catheter assembly 38 and moving the advancement element 22 along the slot from the retracted position to the advanced position. In some embodiments, a diameter of an opening in the distal tip 44 may be less than the second outer diameter such that the second section 32 is prevented from moving through the opening.

As illustrated in FIG. 1E, in some embodiments, a distal end of the coil 24 may include a flat tip 60. As illustrated in FIG. 1F, in some embodiments, the distal end of the coil 24 may include a rounded tip 62. In some embodiments, the flat tip 60 and the rounded tip 62 may decrease a likelihood of the coil 24 damaging the vasculature when the advancement element 22 is moved from the retracted position to the advanced position and the tube 20 and the coil 24 enter the vasculature.

Referring now to FIG. 2, a schematic diagram of the blood collection device 10 is illustrated, according to some embodiments. In some embodiments, when the advancement element 22 is in the retracted position, the tube 20 may extend through the septum 25 (see FIG. 1B) and the coil 24 may be disposed distal to the septum 25. In these embodiments, the septum 25 may be disposed proximal to the position of the septum 25 illustrated in FIG. 1B, such that the coil 24 may still be in the housing 12 when the advancement element 22 is in the retracted position.

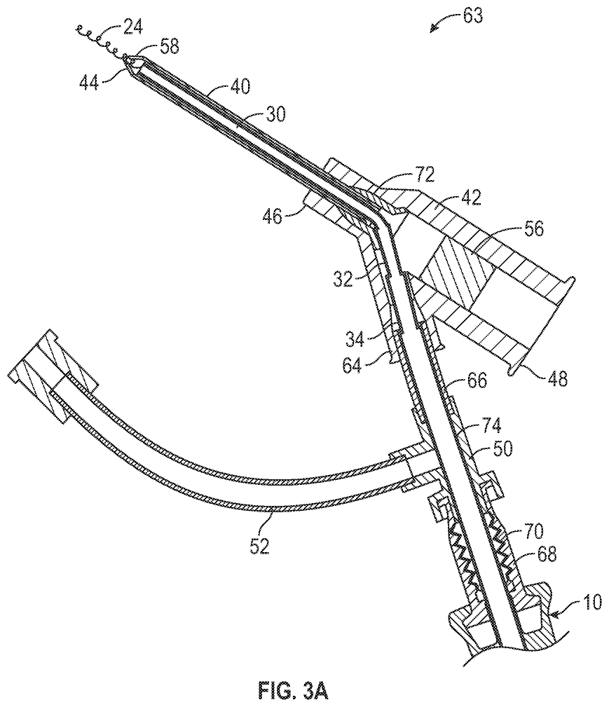

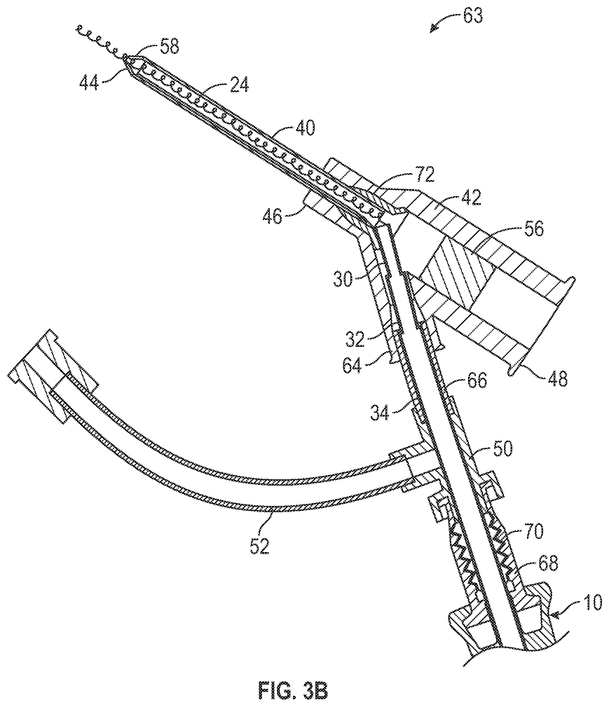

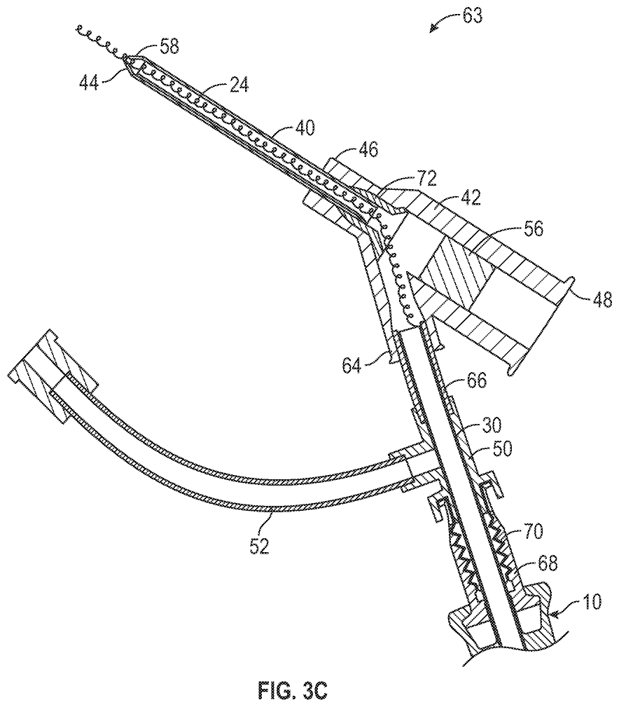

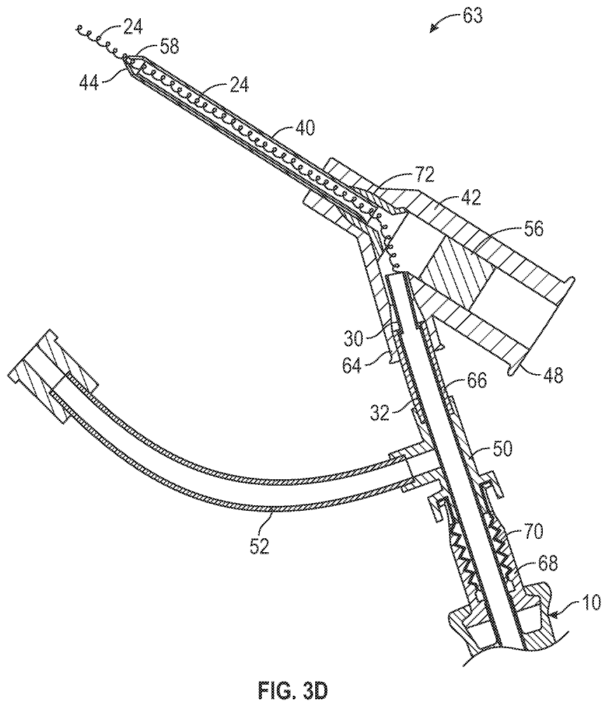

Referring now to FIGS. 3A-3D, a catheter assembly 63 is illustrated, according to some embodiments. In some embodiments, the catheter assembly 63 may be similar or identical to the catheter assembly 38 in terms of one or more components and/or operation. In some embodiments, the blood collection device 10 may be coupled to the catheter assembly 63 in a similar fashion as the catheter assembly 38.

In some embodiments, the catheter assembly 63 may include the catheter adapter 42, which may include the distal end 46, the proximal end 48, and the lumen 54 extending through the distal end 46 of the catheter adapter 42 and the proximal end 48 of the catheter adapter 42. In some embodiments, the catheter adapter 42 may include a side port 64 between the distal end 46 and the proximal end 48 and in fluid communication with the lumen 54. In some embodiments, the catheter assembly 63 may include an extension tube 66, which may include a distal end integrated within the side port 64.

In some embodiments, a proximal end of the extension tube 66 may be integrated with or coupled to the T-connector 50 or another suitable connector. In some embodiments, a needleless access connector 68 that include a septum 70 may be disposed between the T-connector 50 or the other suitable connector and the blood collection device 10. In some embodiments, the needleless access connector 68 may be directly connected to the blood collection device 10.

In some embodiments, a wedge 72 may be disposed within the catheter adapter 42 and may secure the catheter 40 within the catheter adapter 42. In some embodiments, the wedge 72 may include a funnel shape. In some embodiments, the catheter 40 may be engaged in an interference fit with the wedge 72.

In some embodiments, the tube 20 may include a fourth section 74, as illustrated, for example, in FIG. 3A. In some embodiments, the fourth section 74 may include a fourth inner diameter and a fourth outer diameter. In some embodiments, the fourth inner diameter and the fourth outer diameter may be constant along the fourth section 74, such as along an entirety of the fourth section 74.

A position of a distal end of the first section 30 may vary when the tube 20 is fully advanced to facilitate improved blood flow and faster blood collection. In some embodiments, the first outer diameter may be sized to be equal to or slightly less than a particular inner diameter of the catheter assembly 38 where the distal end of the first section 30 is positioned when the tube 20 is fully advanced. Similarly, in some embodiments, the second outer diameter may be sized to be equal to or slightly less than a particular inner diameter of the catheter assembly 38 where a distal end of the second section 32 is positioned when the tube 20 is fully advanced. Similarly, in some embodiments, the third outer diameter may be sized to be equal to or slightly less than a particular inner diameter of the catheter assembly 38 where a distal end of the third section 34 is positioned when the tube 20 is fully advanced.

Similarly, in some embodiments, the fourth outer diameter may be sized to be equal to or slightly less than a particular inner diameter of the catheter assembly 38 where a distal end of the fourth section 74 is positioned when the tube 20 is fully advanced. Increasing or maximizing one or more of the first outer diameter, the second outer diameter, the third outer diameter, and the fourth outer diameter may allow the first inner diameter, the second inner diameter, the third inner diameter, and the fourth inner diameter, respectively, to be increased, thereby increasing blood flow.

As illustrated in FIG. 3A, in some embodiments, in response to movement of the advancement element 22 along the slot 18 (see FIGS. 1A-1B, for example) from the retracted position to the advanced position, a distal end of the first section 30 may be disposed within the catheter 40. In some embodiments, in response to movement of the advancement element 22 along the slot 18 from the retracted position to the advanced position, the first section 30 may contact the distal taper 58. In these embodiments, the first outer diameter may be slightly less than an inner diameter of the catheter 40. In some embodiments, the first outer diameter may contact the inner diameter of the catheter 40 but may allow the first section 30 to move within the catheter 40 when the advancement element 22 is moved to the advanced position. In some embodiments, the fourth section 74 may be directly coupled to the advancement element 22 (illustrated in FIG. 1B, for example).

As illustrated in FIG. 3B, in some embodiments, in response to movement of the advancement element 22 along the slot 18 from the retracted position to the advanced position, a distal end of the first section 30 may be disposed within the catheter adapter 42 proximal to the catheter 40. In these embodiments, in response to movement of the advancement element 22 along the slot 18 from the retracted position to the advanced position, the distal end of the first section 30 may be disposed within the wedge 72 proximal to the catheter 40. In these embodiments, the first outer diameter may be slightly less than an inner diameter of the catheter adapter 42 or the wedge 72. In some embodiments, the first outer diameter may contact the wedge 72. In some embodiments, the third section 34 may be directly coupled to the advancement element 22 (illustrated in FIG. 1B, for example).

As illustrated in FIG. 3C, in some embodiments, in response to movement of the advancement element 22 along the slot 18 from the retracted position to the advanced position, the distal end of the first section 30 may be disposed within the extension tube 66. In these embodiments, the first outer diameter may be slightly less than an inner diameter of the extension tube 66. In some embodiments, the first outer diameter may contact the inner diameter of the extension tube 66 but may allow the first section 30 to move into the extension tube 66 when the advancement element 22 is moved to the advanced position. In some embodiments, the first section 30 may be directly coupled to the advancement element 22 (illustrated in FIG. 1B, for example).

As illustrated in FIG. 3D, in some embodiments, in response to movement of the advancement element 22 along the slot 18 from the retracted position to the advanced position, a distal end of the first section 30 may be disposed within the side port 64 distal to the extension tube 66. In these embodiments, the first outer diameter may be slightly less than an inner diameter of the side port 64. In some embodiments, the first outer diameter may contact the inner diameter of the side port 64 but may allow the first section 30 to move into the side port 64 when the advancement element 22 is moved to the advanced position. In some embodiments, the second section 32 may be directly coupled to the advancement element 22 (illustrated in FIG. 1B, for example).

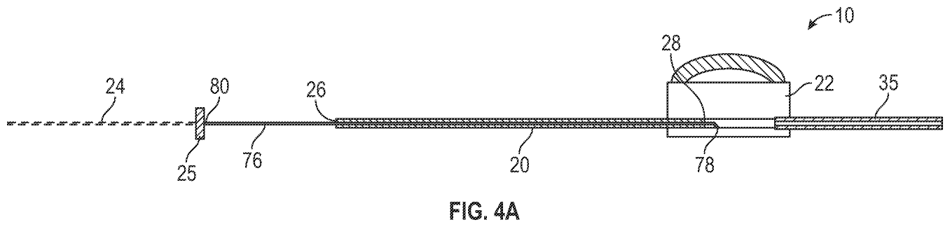

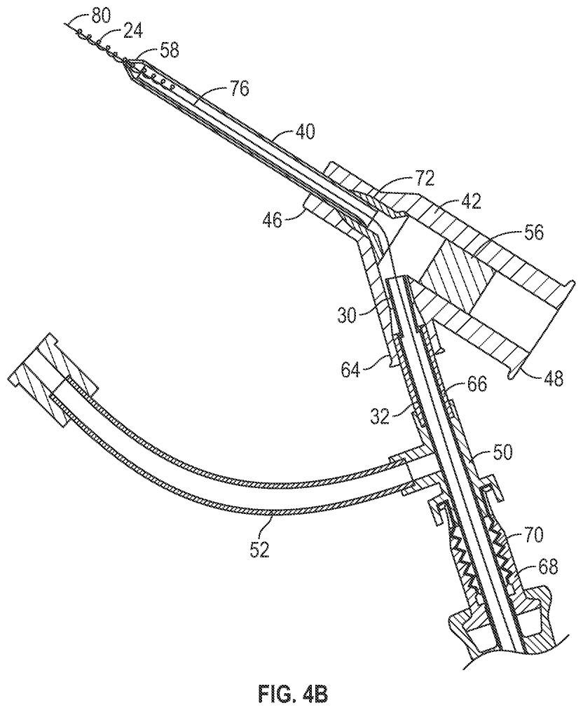

Referring now to FIG. 4A, a schematic diagram of the blood collection device 10 is illustrated, according to some embodiments. In some embodiments, when the advancement element 22 is in the retracted position, the tube 20 may not extend through the septum 25 and/or the coil 24 may be disposed distal to the septum 25. In these embodiments, the tube 20 may be have a large diameter and a shortened length, which may provide improved blood flow and blood collection rates and reduce a risk of infusate and saline contamination. When the advancement element 22 is in the retracted position, the distal end of the first section 30 may be within the extension tube 66 in these embodiments, as illustrated, for example, in FIG. 3C. In some embodiments, the septum 25 may be disposed proximal to the position of the septum 25 illustrated in FIG. 1B, such that the coil 24 may still be in the housing 12 when the advancement element 22 is in the retracted position.

Referring now to FIGS. 4A-4B, in some embodiments, the blood collection device 10 may include a core wire 76, which may include a proximal end 78 and a distal end 80. In some embodiments, the core wire 76 may extend through the tube 20. In some embodiments, the distal end 80 may extend through the septum 25 when the advancement element 22 is in the retracted position. In some embodiments, the proximal end 78 of the core wire 76 may be secured within the advancement element 22. In some embodiments, the core wire 76 may be coupled to a proximal end of the coil 24 and/or extend through the coil 24. In some embodiments, the coil 24 may include the flat tip 60 or the rounded tip 62 (see FIGS. 1E-1F) or another atraumatic tip. In some embodiments, the core wire 76 may be generally straight and/or cylindrical. The core wire 76 may be used with any of the tubes 20 of FIGS. 3A-3D, for example.

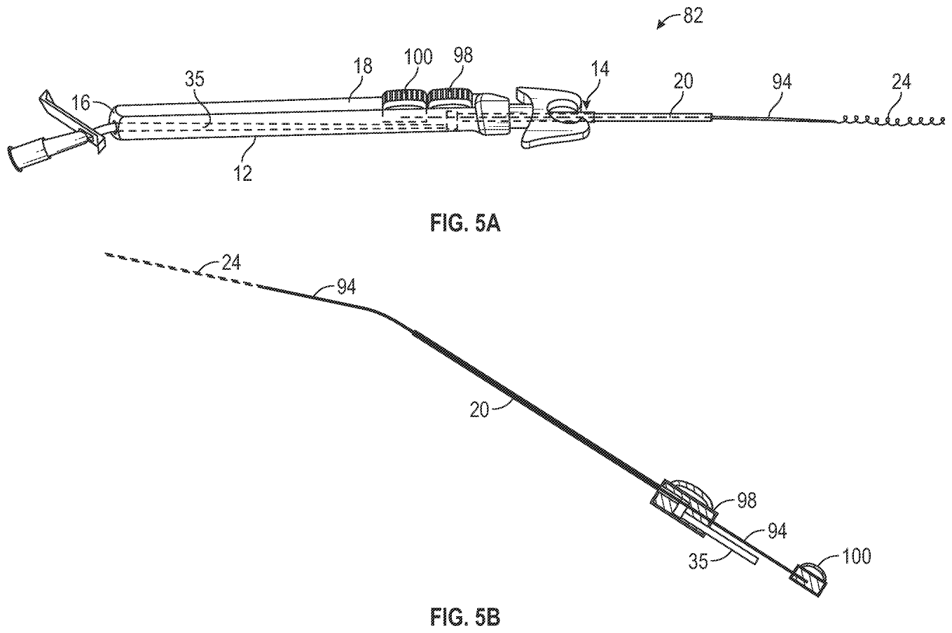

Referring now to FIGS. 5A-5B, a blood collection device 82 is illustrated, according to some embodiments. In some embodiments, the blood collection device 82 may be similar or identical to the blood collection device 10 in terms of one or more components and/or operation. In some embodiments, the blood collection device 82 may be configured to couple to the catheter assembly 38 (see FIG. 1C), the catheter assembly 63 (see FIGS. 3A-3D), or another suitable catheter assembly.

In some embodiments, the blood collection device 82 may include the housing 12, which may include the distal end 14, the proximal end 16, and the slot 18. In some embodiments, the blood collection device 82 may include the tube 20 disposed within the housing 12 and a guidewire 94 comprising the coil 24. In some embodiments, a distal advancement element 98 may be configured to move along the slot 18 and may be coupled to the tube 20. In some embodiments, in response to distal movement of the distal advancement element 98 along the slot 18, the tube 20 may be configured to advance distally beyond the distal end 14 of the housing 12.

In some embodiments, a proximal advancement element 100 may be configured to move along the slot 18 and may be coupled to the guidewire 94. In some embodiments, in response to distal movement of the proximal advancement element 100 along the slot 18, the coil 24 may be configured to advance distally beyond the distal end 14 of the housing 12.

Referring now to FIG. 5B, in some embodiments, when the distal advancement element 98 and the proximal advancement element 100 are each in an initial position, the distal advancement element 98 may be distal and spaced apart from the proximal advancement element 100. In some embodiments, a height of the proximal advancement element 100 may be less than a height of the distal advancement element 98 and/or the proximal advancement element 100 may not span a lumen of the housing 12 such that a space is created between the proximal advancement element 100 and the housing 12. In some embodiments, the extension tube 35 may extend from the distal advancement element 98 through the space. In other embodiments, the extension tube 35 may extend from the distal advancement element 98 through the proximal advancement element 100 and may be movable with respect to the proximal advancement element 100.

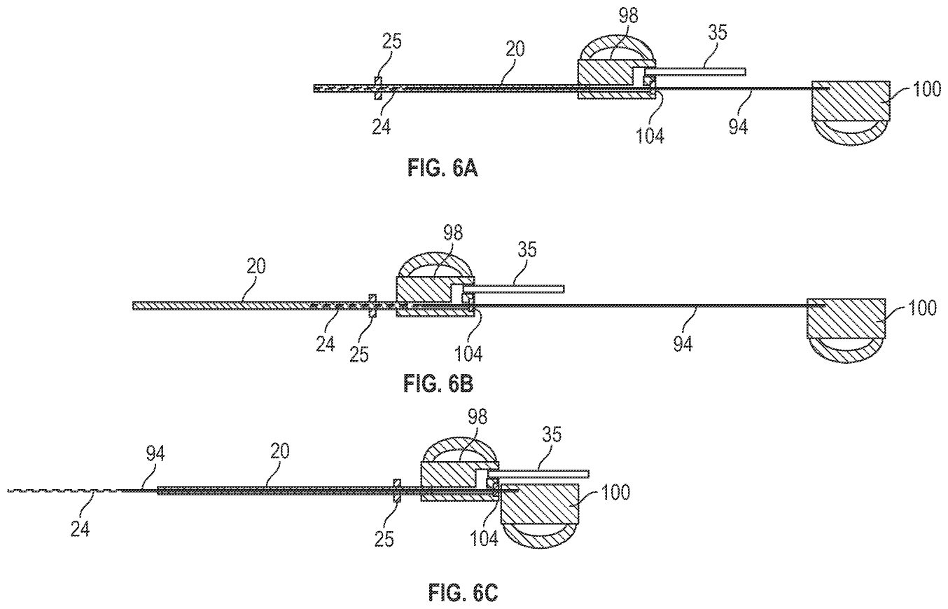

Referring now to FIGS. 6A-6C, in some embodiments, the distal advancement element 98 may be moved from a retracted position to an advanced position before the proximal advancement element 100 is moved or advanced. In other embodiments, the distal advancement element 98 and the proximal advancement element 100 may be proximate each other and advanced together from a retracted to an advanced position. In some embodiments, the distal advancement element 98 and the proximal advancement element 100 may couple together in response to the distal advancement element 98 and the proximal advancement element 100 contacting each other, which may facilitate a single motion retraction.

In FIG. 6A, the distal advancement element 98 and the proximal advancement element 100 are each illustrated in an initial or retracted position, according to some embodiments. In some embodiments, the distal advancement element 98 may include a septum 104 through which the guidewire 94 may extend. In some embodiments, when the distal advancement element 98 is in the initial or retracted position, the distal advancement element 98 may be disposed between a distal end and a proximal end of the slot. In some embodiments, when the proximal advancement element 100 is in the initial or retracted position it may be disposed at the proximal end of the slot 18.

In FIG. 6B, the distal advancement element 98 is illustrated in an advanced position and the proximal advancement element 100 is illustrated in the initial or retracted position, according to some embodiments. In FIG. 6C, the distal advancement element 98 and the proximal advancement element 100 are each illustrated in the advanced position. In some embodiments, when the distal advancement element 98 and the proximal advancement element 100 are each in the advanced position, the distal advancement element 98 and the proximal advancement element 100 may be disposed at the distal end of the slot 18.

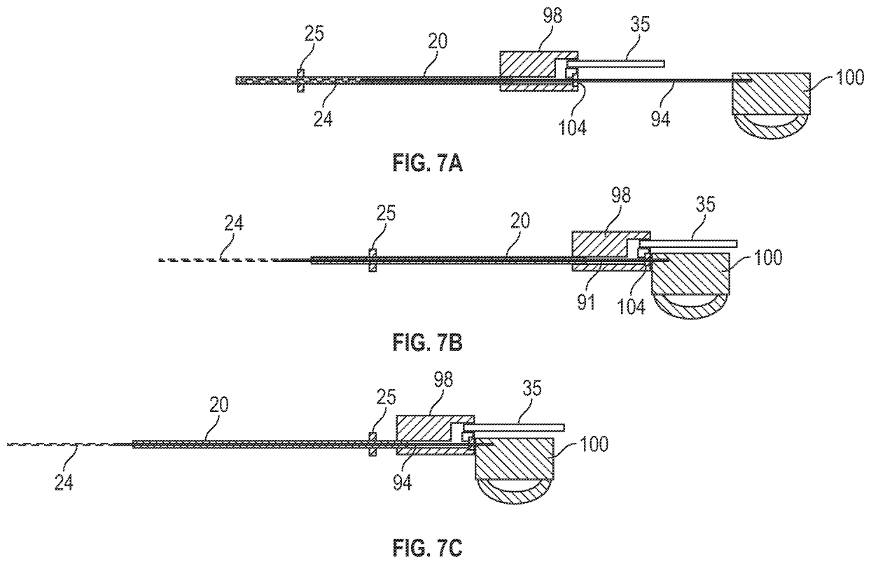

Referring now to FIGS. 7A-7C, in some embodiments, the proximal advancement element 100 may be advanced before the distal advancement element 98, which may advance in response to contact by the proximal advancement element 100. In some embodiments, the proximal advancement element 100 may be configured to move distally along the slot 18 a first distance from the initial or retracted position to contact the distal advancement element 98. In some embodiments, in response to movement of the proximal advancement element 100 distally along the slot 18 a second distance greater than the first distance from the initial or retracted position, the distal advancement element 98 and the proximal advancement element 100 may move distally along the slot 18 together. In these embodiments, the distal advancement element 98 may not include a push tab or handle.

In FIG. 7A, the distal advancement element 98 and the proximal advancement element 100 are each illustrated in an initial or retracted position, according to some embodiments. In FIG. 7B, the proximal advancement element 100 is illustrated in an advanced position and the distal advancement element 98 is illustrated in the initial or retracted position, according to some embodiments. In FIG. 7C, the distal advancement element 98 and the proximal advancement element 100 are each illustrated in the advanced position.

In some embodiments, the tube 20 may include the sections similar to one or more of FIGS. 1-4. In some embodiments, in response to distal movement of the distal advancement element along the slot 18, the first section 30, the second section 32, and/or one or more other sections may be configured to advance distally beyond the distal end 14 of the housing 12, to move outside of the housing 12.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the disclosure.

What is claimed is:

1. A blood collection device configured to couple to a catheter assembly, the blood collection device comprising:
a housing, comprising a distal end, a proximal end, and a slot;
a tube disposed within the housing;
an advancement element configured to move along the slot, wherein in response to distal movement of the advancement element along the slot, the tube may be configured to advance distally beyond the distal end of the housing; and
a coil extending distal to the tube.

2. The blood collection device of claim 1, wherein the coil is coupled to a distal end of the tube.

3. The blood collection device of claim 1, further comprising a core wire disposed within the tube, wherein the core wire is coupled to the coil and extends proximally from the coil to the advancement element.

4. The blood collection device of claim 3, wherein the coil extends into the tube.

5. The blood collection device of claim 3, wherein a proximal end of the coil is distal to a distal end of the tube, wherein a distal end of the core wire is distal to the distal end of the tube.

6. The blood collection device of claim 1, wherein the tube comprises a plurality of sections, wherein the plurality of sections comprises a first section and a second section proximal to the first section, wherein the first section is a distalmost section of the plurality of sections, wherein the first section comprises a first inner diameter and a first outer diameter that are constant along the first section, wherein the second section comprises a second inner diameter and a second outer diameter that are constant along the second section, wherein the coil extends distal to the first section.

7. The blood collection device of claim 6, wherein a proximal end of the coil is coupled to the first section.

8. The blood collection device of claim 6, further comprising a core wire disposed within the tube, wherein the core wire is coupled to the coil and extends proximally from the coil to the advancement element.

9. The blood collection device of claim 8, wherein the coil extends into the tube.

10. The blood collection device of claim 8, wherein a proximal end of the coil is distal to a distal end of the tube, wherein a distal end of the core wire is distal to the distal end of the tube.

11. A catheter system, comprising:
a blood collection device, comprising:
a housing, comprising a distal end, a proximal end, and a slot;
a tube disposed within the housing;
an advancement element configured to move along the slot from a retracted position to an advanced position, wherein in response to movement of the advancement element along the slot from the retracted position to the advanced position, the tube is configured to advance distally beyond the distal end of the housing; and
a coil extending distal to the tube; and
a catheter assembly, comprising:
a catheter, comprising a distal tip and a proximal end.

12. The catheter system of claim 11, wherein the tube comprises a plurality of sections, wherein the plurality of sections comprises a first section and a second section proximal to the first section, wherein the first section is a distalmost section of the plurality of sections, wherein the first section comprises a first inner diameter and a first outer diameter that are constant along the first section, wherein the second section comprises a second inner diameter and a second outer diameter that are constant along the second section, wherein the coil extends distal to the first section, wherein in response to movement of the advancement element along the slot from the retracted position to the advanced position, first section and the second section are configured to advance distally beyond the distal end of the housing.

13. The catheter system of claim 12, wherein in response to movement of the advancement element along the slot from the retracted position to the advanced position, the first section extends through the distal tip and the second section is at least partially disposed within the catheter.

14. The catheter system of claim 12, wherein the blood collection device comprises a septum, wherein in response to movement of the advancement element along the slot from the retracted position to the advanced position, the first section and the second section move through the septum.

15. The catheter system of claim 12, wherein the catheter assembly further comprises:
a catheter adapter, wherein the catheter adapter comprises a distal end, a proximal end, a lumen extending through the distal end and the proximal end, and a side port between the distal end in fluid communication with the lumen; and
an extension tube integrated within the side port, wherein in response to movement of the advancement element along the slot from the retracted position to the advanced position, a distal end of the first section is disposed within the catheter.

16. The catheter system of claim 12, wherein the catheter assembly further comprises:
a catheter adapter, wherein the catheter adapter comprises a distal end, a proximal end, a lumen extending through the distal end and the proximal end, and a side port between the distal end in fluid communication with the lumen; and
an extension tube integrated within the side port, wherein in response to movement of the advancement element along the slot from the retracted position to the advanced position, a distal end of the first section is disposed within the catheter adapter proximal to the catheter.

17. The catheter system of claim 16, wherein the catheter assembly further comprises a wedge securing the catheter within the catheter adapter, wherein in response to movement of the advancement element along the slot from the retracted position to the advanced position, wherein the distal end of the first section is disposed within the wedge proximal to the catheter.

18. The catheter system of claim 12, wherein the catheter assembly further comprises:
a catheter adapter, wherein the catheter adapter comprises a distal end, a proximal end, a lumen extending through the distal end and the proximal end, and a side port between the distal end in fluid communication with the lumen; and
an extension tube integrated within the side port, wherein in response to movement of the advancement element along the slot from the retracted position to the advanced position, a distal end of the first section is disposed within the extension tube or within the side port distal to the extension tube.

19. A blood collection device, comprising configured to couple to a catheter assembly, the blood collection device comprising:
a housing, comprising a distal end, a proximal end, and a slot;
a tube disposed within the housing;
a guidewire comprising a coil;
a distal advancement element configured to move along the slot and coupled to the tube, wherein in response to distal movement of the distal advancement element along the slot, the tube is configured to advance distally beyond the distal end of the housing; and
a proximal advancement element configured to move along the slot and coupled to the guidewire, wherein in response to distal movement of the proximal advancement element along the slot, the coil is configured to advance distally beyond the distal end of the housing.

20. The blood collection device of claim 19, wherein when the distal advancement element and the proximal advancement element are each in an initial position, the distal advancement element is distal and spaced apart from the proximal advancement element.

21. The blood collection device of claim 20, wherein the proximal advancement element is configured to move distally along the slot a first distance from the initial position to contact the distal advancement element, wherein in response to movement of the proximal advancement element distally along the slot a second distance greater than the first distance from the initial position, the distal advancement element and the proximal advancement element move distally along the slot together.

22. The blood collection device of claim 19, wherein the tube further comprises a plurality of sections, wherein the plurality of sections comprises a first section and a second section proximal to the first section, wherein the first section is a distalmost section of the plurality of sections, wherein the first section comprises a first inner diameter and a first outer diameter that are constant along the first section, wherein the second section comprises a second inner diameter and a second outer diameter that are constant along the second section, wherein in response to distal movement of the distal advancement element along the slot, the first section and the second section are configured to advance distally beyond the distal end of the housing.
